(19) United States
(12) Patent Application Publication
(10) Pub. No.: US 2005/0076917 A1
(43) Pub. Date: Apr. 14, 2005

(54) Lubricated condom

(75) Inventors: Daniel X. Wray, Vista, CA (US); Robert L. De Armond, Temecula, CA (US)

Correspondence Address: PROCOPIO, CORY, HARGREAVES & SAVITCH LLP, 530 B STREET, SUITE 2100, SAN DIEGO, CA, 92101, US

(73) Assignee: BIOFILM IP, LLC.

(21) Appl. No.: 10/683,251

(22) Filed: Oct. 10, 2003

(51) Int. Cl. B05D 3/00; B32B 5/16; B32B 27/02; A61L 2/00

(52) U.S. Cl. 128/844; 427/2.1; 428/403; 428/407

(57) ABSTRACT

A rolled-up lubricated condom formed by depositing a water soluble lubricant to the rim of a rolled up condom after a surface of the condom has been coated with a surfactant modified powder. Surfactant modified powder, methods of making surfactant modified powder, and methods of preparing a condom with surfactant modified powder.

### FIELD OF THE INVENTION

The present invention relates to condoms lubricated with water soluble lubricants, compositions of modified powders for preparing lubricated condoms, and methods of making modified powders, and methods of preparing lubricated condoms.

### BACKGROUND

Condoms are sold in envelopes or foils as sheaths which are rolled up or otherwise compacted for storage.

Condoms in the form of a sheath, are worn over the penis to help prevent pregnancy and/or transmission of sexually transmitted diseases such as syphilis, gonorrhea, chlamydia infections, genital herpes and AIDS, for example, during sexual intercourse. Condoms are typically composed of thin barrier membranes of latex or similar elastomeric materials for optimizing sensitivity, tactile sensation and heat transference during intercourse. Without adequately distributed lubrication, the thinness and dry elasticity of condoms, however, present a risk of a break or tear in the condom during sexual intercourse, which is always a significant concern in condom design and manufacturing. Because of the increasing reliance on latex condoms as a barrier against HIV and other sexually transmitted diseases, it is imperative to take every measure to enhance the quality of these products to provide as much disease protection as modern technology will allow. Current condom manufacturing technology applies a dusting powder to a condom that serves to prevent the latex from sticking to itself after the condom is rolled. The rolled and dusted condom is placed in a foil pouch to which a lubricant is added. The dusting of a condom with a powder creates a barrier that prevents a water based lubricant from fully wetting the length of the condom. The result is a condom that is lubricated only 2 to 3 inches of its full length.

### SUMMARY

It has now been unexpectedly discovered that application of a lubricant to a rolled condom, i.e. a contraceptive sheath, which has been coated with a surfactant-modified powder (SMP), substantially coats the condom throughout its length with a lubricant composition in an effective and reliable manner

The present invention is directed to methods of manufacturing a condom which is lubricated substantially the entire length the condom's inner and/or outer surface without directly applying the lubricant composition along the entire body of the prophylactic prior to wrapping for use. The method involves preparing a condom by coating a surface of the condom with a surfactant modified powder. Included is a method of making a composition of a surfactant modified powder, which involves a step of coating a powder with an aqueous composition of surfactant.

The invention is directed to the products of the methods disclosed herein. Included is a composition which comprises a powder which bears a coating of an aqueous composition of surfactant. A condom which bears on its surface a coating of the surfactant-modified powder is a feature of the invention, as well as a condom having a surface coated with a surfactant-modified powder and an aliquot of a water-soluble lubricant deposited on the surface. The condoms of the invention are further disclosed as being encased in an air-tight container.

According to the present invention, a method for manufacturing a lubricated prophylactic includes the steps of dipping mandrels of predetermined size and shape into a molten bath of elastomeric material, withdrawing the mandrels from the bath; allowing the elastomeric material on the mandrels to harden and dry, and coating one or both of the inner and outer surfaces of the condom with surfactant modified powder (SMP). The SMP-coated condom is rolled, and an aliquot of water-soluble lubricant is deposited on the rolled up condom, in contact with the SMP-coated surface(s). The condom is sealed in an airtight container, and an effective amount of the lubricant migrates or spreads to coat the entire rolled up surface(s) of the condom's sheath.

### DESCRIPTION OF DRAWINGS

FIG. 1 is a perspective view of a condom on which surfactant modified powder is disposed or coated on the outer surface.

FIG. 2 is an elevational view through the closed end of a rolled condom.

FIG. 3 is a diagrammatic representation of a partial cut-away of a rolled condom.

FIG. 4 is an elevational view of a rolled up condom encased in an air-tight container.

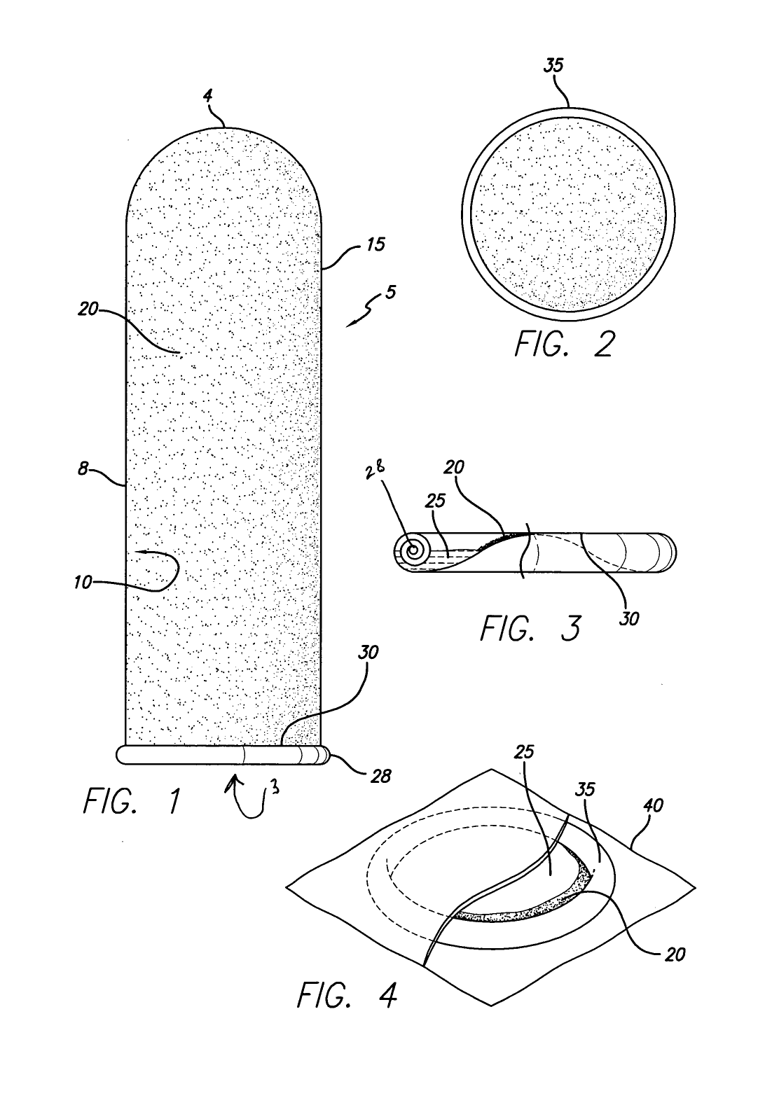

### DETAILED DESCRIPTION OF THE INVENTION

In one aspect, the present invention is directed to a lubricated condom. The lubricated condom is formed from a condom 5 which, prior to being rolled up for storage, has been coated on its inner 10 and/or outer 15 surfaces with surfactant-modified powder (SMP) 20. An aliquot 25 of water-soluble lubricant is applied to the inner rim 30 of the rolled up condom 35. After a sufficient time, the water-soluble lubricant migrates through the SMP powder on the SMP-treated surface of the condom, ultimately wet-lubricating the entire length of the SMP-treated surface of the condom.

As used herein, and as recited in the U.S. Manual of Patent Classification 604/349, a condom refers to a receptacle structured for collecting semen from a penis. The subject condom refers to a receptor which is shaped and designed so as to fit around the penis to receive emitted semen. More particularly, a condom is a flexible sheath which defines an open end 3, a closed end 4, and an elongate wall portion 8, having inner 10 and outer 15 surfaces, generally in the shape of a tube extending between the open and closed ends. The sheath is manufactured and sold in a rolled configuration 35. When used to sheath the penis, the condom is unrolled. A condom is typically made of thin, flexible, natural or synthetic rubber material for use during sexual intercourse to prevent disease and to receive semen.

The Pre-Lubricated Condom Manufacturing Process

It is well-known in the industry that a pre-lubricated condom (i.e., a packaged, rolled-up condom 40 which already has lubricant on its exterior surfaces) is usually the consumer's product of choice. Rolled up in the conventional manner, the generally tubular or cylindrical wall of condom comprises a lubricated exterior surface.

Condoms are manufactured in a variety of processes which generally involve plural sequential shaping or molding steps on same workpiece. The process involves incremental molding or accretion from bulk, e.g., successive dip casting, and those operations which involve repetition to give an overall unitary effect.

Traditional methods of manufacturing a contraceptive prophylactic involve dipping a phallic shaped ceramic mandrel (which may be stationary or rapidly spinning about its longitudinal axis) of predetermined size into a warm bath containing molten elastomeric material suitable for use as a condom. The liquid cover material coats the mold projections and is allowed to harden. The immersion of the mandrel with the distal end first into, say, latex solution results in the formation of a condom having a wall which has an inner and outer surface.

Condoms are made of an elastic material, such as latex, and are packaged individually, with or without a lubricant or wetting agent, in a sealed plastic or foil pouch having an upper and a lower generally flat surface. When packaged, the condom is rolled in a manner such that it appears as a latex disk with a tubular edge. Sometimes, during further processing and testing of the condom it may be placed on several different mandrels by unrolling it onto the mandrel and then rolling it to remove it from the form.

Before being rolled up for packaging, the finished condom is coated with powder, typically starch powder, which prevents the surfaces of the condom from adhering to each other during storage. A widely used method of “dusting” condoms involves immersing them in a powdering tank (a slurry consisting of corn starch, magnesium carbonate, and anti-bacterial chemicals). This process keeps the latex from sticking.

After coating a surface with powder, the sheath is rolled around a thickened elastic ring, which is formed at the open end of the condom, to form a cup 26 within the circumference of the ring 28. The results in several layers of elastomeric material being rolled around the thickened ring formed at the open end of the sheath, with the closed end of the sheath forming a cup within the circumference of the ring. In this form, lubricants may be added to the cup-shaped elastomer sheath, i.e. to the inside of the peripheral part of the rolled condom. The lubricant solution is intended to gradually permeate so as to spread uniformly over the whole surface of the condom to give a comfortably moist and supple coated condom. However, it is well known that a substantial length of the distal the outer surface of pre-packaged lubricated condoms remains unlubricated.

Referring to FIGS. 2 and 3, the rim 28 or ring at the open end of condom is substantially one to fifteen very tight convolutions of sheath material. During manufacture of the condom, when the freshly-formed sheath is still on the dipping mandrel (not shown), rolling of the rim in the outward direction (i.e., outwardly onto the outer surface of the condom) removes the condom from the dipping mandrel. During this rim-rolling step, the interior surface is rolled against a powder treated outer surface, which prevents the condom from adhering to itself.

The rolled up sheaths are sealed in individual foil wrappers 40. The condom may be foiled dry, or lubricated with silicone or an appropriate anti-friction agent, and/or with a spermicidal agent for added protection against pregnancy.

For example, without limitation, U.S. Pat. No. 4,684,490 teaches a method for manufacturing condoms in which a mandrel having the general shape and dimensions of a condom is dipped into a solvent solution of a polyurethane polymer and heated in air after being withdrawn therefrom so as to dry the polyurethane. The dried polyurethane which now forms a condom is then removed from the mandrel.

Use of Surfactant-Modified Powder for Pre-Lubricating Condoms

The methods of the present invention involve well-known methods of making sheath-type condoms, which are manufactured through a variety of techniques well known to those skilled in the art of condom manufacturing, the most widely used technique being the dip-molding process, wherein a core or mandrel is dipped in a liquid-state dipping solution. The core becomes covered with a thin film of polymer which can, if desired, be further coalesced and/or cross-linked or vulcanized with heat or chemical treatments.

Condoms of this invention can be manufactured using a variety of techniques, including dip-molding (dip-coating), spray-forming, vacuum-forming, heat sealing, and blow-molding, the ordinarily preferred technique being a modification of conventional dip-coating, since most condom manufacturing facilities utilize dip-coating equipment.

The basic sheath material can, if desired, be formed from natural or synthetic rubber lattices, synthetic polyisoprene, polyurethane, polyurethane-urea, poly (siloxane urethane/urethane-urea) copolymers, poly(1,4 diene-vinyl aryl) random and block copolymers, or other suitable polymeric materials capable of forming films. Natural rubber latex is desirable in terms of its strength, modulus, extensibility, etc., its ready availability (particularly in the condom industry), and its high coefficient of friction at the surface.

The methods of the invention employ the above-described techniques and materials for making packaged condoms, except that the powder employed in the step of coating or dusting a surface of the condom is replaced with surfactant-modified powder.

Regardless of the method employed, a result is a rolled-up condom which comprises inner and/or outer surfaces coated with surfactant-modified powder. Ultimately, an aliquot 25 of water-soluble lubricant is applied to the inner rim 30 or cup 26 of a rolled-up condom in contact with an SMP-treated surface. After packaging and storage in an air-tight container, when the rolled-up sheath is unrolled (e.g., prior to intercourse), its surface(s) are coated with water-soluble lubricant.

The cup shaped latex sheath is now ready for application of the lubricant composition. At this point, a measured aliquot 25 of lubricant agent is dispensed (e.g. from an overhead burette or needle valve) onto the coated surface, preferably at the inner rim 30 of the rolled up condom. The condom is then wrapped in an airtight container (e.g. sealed in an aluminum foil packet) and stored for ultimate use by the consumer. Foiling is the final stage of the manufacturing process in which condoms are carefully lubricated by depositing approximately 1 ml to 3 ml of water-soluble lubricant on the rolled up condom, and enclosing it in aluminum-based foiling. The prophylactic may be conveniently packaged and substantially all of the prophylactic will become covered with lubricant while contained within the airtight (foil) packaging.

Coating a Condom Surface with Surfactant-Modified Powder

It has been discovered that by coating a surface of a condom with SMP powder prior to rolling up the condom, that a water-soluble lubricant deposited in the elastomeric cup will flow substantially over the entire SMP-coated surface of the rolled condom sheath within the airtight package over a period of about eight to fourteen days, thereby producing the lubricated condom of the invention. As such, it is not necessary to coat the condom with lubricant while it is on the mandrel.

Coating a surface of the condom with SMP can be achieved by applying SMP-powder onto either or both of inside and outside surfaces of a condom, or necessary portion of a condom by dropping, dipping, coating or spraying a solution containing SMP to form a coating entirely or partly on the surface of the condom.

Surfactant-Modified Powder

Surfactant modified powder is formed by treating a powder, which is safe and non-toxic for contact with human skin, with a surfactant to form the surfactant modified powder of the invention.

A powder is selected from that group of powders which the industry applies to elastomeric sheath material to prevent the material from adhering to itself. For example, natural rubber latex films have extremely strong adhering tendencies, to the extent that two thin natural rubber latex surfaces placed in contact with each other would be extremely difficult if not impossible to separate. Traditionally, an anti-adherence, friction-reducing material or “dressing agent” (a dusting powder or lubricant or a combination thereof) is applied to both the exterior and interior surfaces of the sheath prior to or during removal from the core or prior to rolling up of the sheath. Typical dusting agents include talc, calcium carbonate, silica, lycopodium, corn or potato starch, or the like, or combinations thereof.

Starch powder is the preferred powder for use in the invention. It is well known in the industry how to dust a surface of a condom with starch powder after a condom mandrel is dipped into a polymer solution and cooled.

It is well known in the art to apply powder to latex or other elastomeric surfaces of wearable articles to make it easier for the user to don the article. The art teaches that during processing, the “outside” of a condom is coated with a donning lubricant. In one mode of a condom manufacturing process, a condom is inverted when it is stripped from the mandrel. For most powdered condoms, the “outside” lubricant is cornstarch which remains, after stripping, inside the inverted condom as the donning lubricant or powder.

Donning lubricants, such as cornstarch, silicone, etc., are used to ease insertion of the users penis into the condom. Powdered lubricants are also called donning powders or dusting powders. Cornstarch which meets the specification for absorbable donning or dusting powder in the United States Pharmacopeia (U.S.P.) is a commonly used lubricant for condoms. Talc, cotton flock, and other non-absorbable materials are less preferable as a lubricating, dusting, or donning powder.

A preferable absorbable powder is defined as a powder made from corn starch that meets the specifications for absorbable powder in the U.S.P., and, which, in other applications, is intended to be used to lubricate the penis in order to ease the donning of the condom.

The physical and chemical properties and specifications of the absorbable dusting powder useful in the invention involve size and size distribution of the powder particles. The specifications for an absorbable dusting powder should include aspects such as those described in U.S.P. for corn starch-based absorbable dusting powder, including specifications for the upper and lower limits for the degree of cross-linking.

Absorbable dusting powder is a specially processed cornstarch. It is a substance recognized in the United States Pharmacopeia-National Formulary (USP-NF). The USP-NF is a standards setting body in the U.S. The USP-NF is officially recognized in the Federal Food, Drug and Cosmetic Act.

U.S. Pat. No. 5,405,666 teaches achieving conventional dry slip by the use of powder lubricants such as magnesium carbonate, starch and talc. The particles can be cross-linked corn starch, nylon, polyurethane or mixtures and should be of a size range between 5 and 50 microns with an average size between 30 and 40 microns.

Suitable particles include microparticles of silica, polyamide or cellulose triacetate particles having a size within the range of about 5 to about 13 microns and a relatively narrow size distribution. Preferably, the particles have a substantially regular shape without sharp angles or edges and are in substantially nonaggregated form (e.g., are primary particles of a near-spherical shape).

Other powders or dusting agents suitable for modification by surfactant include certain alkali metal compounds (U.S. Pat. No. 4,143,423, incorporated by reference.), which are nontoxic and which do not cause adverse reactions in animals or human patients. These compounds include a chemical compound of one alkali metal selected from the group consisting of sodium (Na) and potassium (K) in chemical combination with a radical selected from the group consisting of carbonate (CO₃), bicarbonate (HCO₃), acetate (C₂.H₃O₃), acetate trihydrate (C₂ H₃ O₂.3H₂O), and citrate dihydrate (C₆ H₅O₇.2H₂O). Examples of other chemical compounds which may be used as the nontoxic powder or dusting agents of the present invention include sodium carbonate (Na₂ CO₃), sodium bicarbonate (NaHCO₃), sodium citrate dihydrate (Na₃C₆H₅O₇.2H₂ O), sodium acetate (NaC₂ H₃O₂), sodium acetate trihydrate (NaC₂ H₃O₂.3H₂ O ), potassium carbonate (K₂2 CO₃), potassium bicarbonate (KHCO₃), potassium citrate, dihydrate (K₃3 C₆ H₅ O₇.2H₂O) and potassium acetate (KC₂H₃O₂).

Formulating Surfactant-Modified Powder

Surfactant-modified powder is formulated by applying or contacting powder with an aqueous solution containing from about 0.002% to about 1% surfactant, preferably about 0.25% to about 0.5% surfactant.

The amount of surfactant may vary according to the type of surfactant used, the nature and particle size of the powder. While resulting in adequate lubricity, the amount of surfactant present, however, should be at least sufficient to achieve wetting of a length of the condom in less time than it would take to achieve wetting of a comparable length of normal (i.e. non-surfactant), powder-treated condom.

Thus, according to the present invention the powder comprises from about 0.1 to 5 parts by weight per hundred of surfactant, based on dry powder weight, preferably from about 0.5 to 1.0 parts per hundred by weight.

Surfactant

The surfactant used to coat the powder may be any surfactant which is suitable for use on skin or other tissue and does not cause an allergic, irritant, or other undesirable reaction in said skin or other tissue. Thus amphoteric, anionic, cationic, and nonionic surfactants, and long-chain fatty amines can be used, as taught for example in various patents and patent applications recited in U.S. Pat. No. 5,405,666, herein incorporated by reference.

Suitable cationic surfactants include those comprising at least one lipophilic moiety such as an alkyl, aralkyl, aryl, or cycloalkyl group containing 6 to 18 carbon atoms, and a hydrophilic moiety such as a substituted ammonium group (for example, a tetra-alkylammonium, pyridinium, or like group). The counter-ion present should be compatible with the tissue of the wearer; it could be, for example, chloride or other halide. Preferred cationic surfactants are quaternary ammonium compounds having at least one C₈-C₁₈ hydrocarbyl (alkyl, aryl, aralkyl or cycloalkyl) group; a preferred hydrocarbyl group is a hexadecyl group. The hydrocarbyl group may be attached to a quaternary nitrogen atom which is part of a heterocyclic ring (such as a pyridine, morpholine, or imidazoline ring).

Compositions of surfactants useful in the invention may also contain various other agents. One such composition involves water soluble lubricants. A widely marketed class of water soluble lubricants comprise polyquaternium, glycerin and a preservative system. Two commercially available vaginal lubricant products are: AstroGlide(™) (BioFilm, Vista, Calif.) and Vagisil(™) (Combe Inc., White Plains, N.Y.). The AstroGlide(™) composition is also sold under the brands Silken Secret(™), ViAmor(™), and Target(™). The AstroGlide(™) product includes water, glycerin, polyquaternium #33,propylene glycol, methylparaben and propylparaben. Vagisil(™) comprises polyquaternium #32. Without limitation, other polyquaternium-containing vaginal lubricants, included in the surfactant compositions are Just Between Us(™) Personal Lubricant (Key West Aloe Co.),CVS(™) Personal Lubricant (Sun Mar Laboratories), and Ultra Lube(™) (Super Brands), all of which contain polyquaternium #5. Enhance(™) Personal Lubricant (The Xandria Collection) contains polyquaternium #7. Also without limitation, non-polyquaternium containing personal lubricants included in the composition of the methods disclosed herein include Replens(™) Vaginal Moisturizer, K-Y Liquid(™), K-Y7 Long Lasting Vaginal Moisturizer, K-Y Silk-E(™) Vaginal Moisturizer, K-Y(™) Jelly Personal Lubricant, Summer's Eve(™) Vaginal Moisturizer, Aqua Lube(™), and Wet, Light(™)Personal Lubricant Gel.

Concentration ranges of polyquaternium useful in the present invention are from 0.1% to 10% by weight. Alternatively, any polyquaternium can be used at any concentration that is non-irritating to the mucosal epithelium. It is contemplated that the polyquaternium used herein is cationic in nature, although polyquaterniums which are anionic or non-ionic also find use n the compositions of the method. The International Cosmetic Ingredient Dictionary and Handbook (7^{th} Edition (1997),Vol. 2, eds. J. A. Wenninger and G. N. McEwen, publ. The Cosmetic, Toiletry, and Fragrance Association, Wash. D.C.) discloses, without limitation, polyquaterniums (and their commercial sources) that are useful in the compositions of the methods of the invention.

Concentration ranges of glycerol useful in the present invention are from about 5% to about 60% by weight. An example of a concentration range of a preservative system useful in the invention involves methylparaben at 0.03% to 0.3% by weight and propylbaraben at 0.015% to 0.15% by weight.

As used herein, the term “preservative system” refers to a chemical or group of chemicals that act to prevent the contamination or degradation of other components in the composition. For example, methylparaben and propylparaben are used as preservatives herein. Examples of other preservatives include, but are not limited to, FDA approved preservative systems for food, cosmetics, and food preparations.

The coating of surfactant need not coat the powder surface completely. It is only necessary that enough surfactant is applied to improve the rate of lubricant spread on the rolled up, SMP-coated surfaces of a condom..

The invention is not limited to the surfactants hereinbefore described and may also include mixtures of two or more surfactants.

Non-ionic surfactants are useful in the invention, and examples of suitable non-ionic surfactants include those described in “Surfactant and Interfacial Phenomena,” Milton J. Rosen, (John Wiley & Sons, Inc. (1978)). Examples of non-ionic surfactants which may be used are: polyoxyethylene alkylphenols; alkylphenol ethoxylates (examples of alkylphenols are p-nonylphenol, p-octylphenol or p-dodecylphenol; polyoxyethylenated straight chain alcohols); alcohol ethoxylates (the alcohols are typically derived from coconut or tallow oils or are synthetic long carbon chain alcohols); polyoxyethylenated polyoxypropylene glycols (derived from ethylene oxide and propylene oxide); polyoxyethylenated mercaptans; long chain carboxylic acid esters (e.g., glycerol and polyglyceryl esters of natural fatty acids, propylene glycol, sorbitol and polyoxyethylenated sorbitol esters); polyoxyethylene glycol esters and polyoxyethylenated fatty acids; alkanolamine condensates; alkanolamides (e.g., alkanolamine/fatty acid condensates); and tertiary acetylenic glycols.

Water Soluble Lubricants

Water-soluble lubricant agents that are suitable for use in the process and product of the present invention may be generally classified as liquid hydrogels. For the purposes of this invention, a hydrogel is considered to be a polymeric material that can absorb more than 20% its weight in water while maintaining a distinct three-dimensional structure. This definition includes dry polymers that will swell in aqueous environments in addition to water-swollen materials. A hydrogel that dries without significant collapse of the macroscopic structure and which absorbs water into macropores without substantial macroscopic swelling is termed an “aerogel,” while a nonporous gel that absorbs water by swelling is called a “xerogel.” This definition does exclude the Type 1 and 4 gels used in pharmacy, such as the various hydrophilic polymers that are used as binders, disintegrants, and so on, but is consistent with the understanding of gels accepted by the broadest range of polymer scientists. Hydrogels for this invention may be organic or non-organic, although the USP has defined gels as semisolids consisting of inorganic particles enclosed and penetrated by a liquid.

Hydrogels are an exceptionally diverse group of materials. Any technique used to form polymeric networks can be used to produce hydrogels from hydrophilic polymers. Virtually all hydrophilic polymers can be crosslinked to produce hydrogels, whether the polymer is of biological origin, semi-synthetic, or wholly synthetic. Hydrogels can be made biologically inert or biodegradable; they are easily derivatized with specialized groups like enzymes. They can be made in virtually any size or shape using any polymer production technique. Hydrogels can even be made such that their properties continually adjust in response to changes in their environment.

The key properties that make hydrogels thus defined valuable are the equilibrium swelling degree, sorption kinetics, solute permeability, and their in vivo performance characteristics. The equilibrium swelling degree or sorption capacity (swollen volume/dry volume) is the single most important property of a hydrogel and directly influences the other properties. Depending upon the formulation, the swelling degree can be varied virtually without limit. Although the sorption kinetics do depend upon the gel composition, the sorption rate is roughly proportional to the equilibrium swelling degree, and thus is also widely variable. Permeability to water, drugs, proteins, and other solutes can also be varied over extraordinarily broad ranges, again, depending primarily upon the swelling degree or water content. In vivo, hydrogels can be designed to be biocompatible and/or biodegradable. The high water content of hydrogels also enhances their biocompatibility, since impurities can be easily extracted, their soft, flexible nature mimics natural tissue, and their hydrophilic character minimizes interfacial tension.

Hydrogel coating were introduced to the medical device industry in the early 1960s (Wichterle & Lim, 1960), to enhance lubricity. In these applications, the absorbed water in the hydrogel coating forms a thin water film on the contacting surface, fostering lubricity. This allows for device movement. Hydrogels may also be referred to as hydrocolloids, which are hydrophilic polymers, of vegetable, animal, microbial or synthetic origin, that generally contain many hydroxyl groups and may be polyelectrolytes. They are naturally present or added to control the functional properties—viscosity, elasticity—of aqueous compositions for topical application to skin. Most important among these properties are viscosity (including thickening and gelling) and water binding but also significant are many others including emulsion stabilization. The most important of the hydrocolloids are agar, alginate, arabinoxylan, carrageenan, carboxymethylcellulose, cellulose, curdlan, gelatin, gellan, b-glucan, guar gum, gum arabic, locust bean gum, pectin, starch, xanthan gum.

A group of water-soluble lubricants for use in the methods and products of the invention are described above, namely those which contain polyquaternium, glycerin, and a preservative system. Preferred water soluble lubricants are two AstroGlide(™) (BioFilm, Vista, Calif.) and Vagisil(™) (Combe Inc., White Plains, N.Y.). The AstroGlide(™) composition is also sold under the brands Silken Secret(™), ViAmor(™), and Target(™). The AstroGlide(™) product includes water, glycerin, polyquaternium #33, propylene glycol, methylparaben and propylparaben. Vagisil(™) comprises polyquaternium #32. Without limitation, other polyquaternium-containing vaginal lubricants, included in the surfactant compositions are Just Between Us(™) Personal Lubricant (Key West Aloe Co.),CVS(™) Personal Lubricant (Sun Mar Laboratories), and Ultra Lube(™) (Super Brands), all of which contain polyquaternium #5.

Enhance(™) Personal Lubricant (The Xandria Collection) contains polyquaternium #7. Also without limitation, non-polyquaternium containing water-soluble, personal lubricants included compositions useful in the products and methods disclosed herein include Replens(™) Vaginal Moisturizer, K-Y Liquid(™), K-Y(™) Long Lasting Vaginal Moisturizer, K-Y Silk-E(™) Vaginal Moisturizer, K-Y(™) Jelly Personal Lubricant, Summer's Eve(™) Vaginal Moisturizer, Aqua Lube(™), and Wet, Light(™)Personal Lubricant Gel.

### EXAMPLES

### Example 1

A condom coated with a surfactant modified dusting powder increased the amount and rate of lubricious wetting compared to a condom coated with untreated powders.

Condom Coating Process

A wetting solution, which had the following composition, was prepared:

| Components | Concentration | Composition |
|---|---|---|
| Purified water | 49.99 | 49.99 g |
| Polyquaternium #15 | 0.02 | 0.02 g |
| Isopropyl Alcohol | 49.99 | 49.99 g |

A surfactant modified powder, or coating treatment was prepared by mixing the wetting solution with Absorbo(™) HP powder, according to the following concentration:

| Components | Concentration | Composition | |
|---|---|---|---|
| Absorbo Powder | 94.59% | 175 | g |
| Wetting Solution | 5.41% | 10 | mL |

Two teaspoons of the surfactant modified Absorbo(™) powder and five unrolled, uncoated/unlubricated condoms were placed in a motorized tumbler drum. The condoms were tumbled with the surfactant modified powder for 10 minutes. The powdered condoms were removed from the drum, and each one shaken to remove excess powder.

Two teaspoons of the unmodified Absorbo(™) powder and five unrolled, uncoated/unlubricated condoms were placed in a motorized tumbler drum. The condoms were tumbled with the unmodified powder for 10 minutes. The powdered condoms were removed from the drum, and each one shaken to remove excess powder.

Each of the condoms was stretched over a glass condom mold until fully deployed. Working from the open end of the condom, each condom was rolled back unto itself to form a tightly rolled condom with only the tip protruding from the center.

Each condom was placed in a 40 ml beaker with the tip of the condom facing up.

Using a syringe, 5 ml of Astroglide(™) were placed on each condom, ensuring that the Astroglide(™) was captured in the reservoir of the condom. The top of each beaker was sealed with paraffin film.

At various time intervals, condoms were removed from their respective lots, mounted and stretched over a glass condom mold until fully deployed. For each condom, the length migration of lubricant over the sheath of the condom, i.e. the length of the condom which was wetted and lubricious was measured.

In the following table, “untreated condoms” designates condoms which were coated with untreated Absorbo(™) powder; “treated condoms” were coated with surfactant modified powder.

Observations:

| Date | Untreated Condom | Treated Condom |
|---|---|---|
| Aug. 26, 2003 | Condoms placed into solution | |
| Aug. 29, 2003 | Wetted area is lumpy and | Wetted area is smooth and |
| | uneven. (Approx. 2″) | uniform. (Approx. 3″) |
| | Astroglide solution readily | Astroglide solution adheres |
| | runs off the surface of the | to the surface of the |
| | condom | condom with minimal run- |
| | | off. |
| Sep. 02, 2003 | Wetted area is lumpy and | Wetted area is smooth and |
| | uneven. (Approx. 2″) | uniform. (Approx. 4″) |
| | Astroglide solution readily | Astroglide solution adheres |
| | runs off the surface of the | to the surface of the |
| | condom | condom with minimal run- |
| | | off. |
| Sep. 05, 2003 | Wetted area is lumpy and | Wetted area is smooth and |
| | uneven. (Approx. 2″) | uniform. (Approx. 5″) |
| | Astroglide solution readily | Astroglide solution adheres |
| | runs off the surface of the | to the surface of the |
| | condom | condom with minimal run- |
| | | off. |
| | 3.2 mL of Astroglide | 3 mL of Astroglide |
| | remained inside the beaker | remained inside the beaker |
| Sep. 09, 2003 | Wetted area is lumpy and | Entire length of condom is |
| | uneven. (Approx. 2.5″) | wetted. Surface is smooth |
| | | and uniform. (Approx. 7″) |
| | Astroglide solution readily | Astroglide solution adheres |
| | runs off the surface of the | to the surface of the |
| | condom | condom with minimal run- |
| | | off. |
| | 4 mL of Astroglide | 2.8 mL of Astroglide |
| | remained inside the beaker | remained inside the beaker |

In all instances, the minimum advance of the lubricant along the prophylactic sheath increased with time. This indicates that the lubricant is advancing along the length of the latex sheath. The maximum penetration figure of the treated condoms appears to advance gradually fairly quickly to approximately the full length of the prophylactic. This example demonstrated a method for making a surfactant coated dusting powder that, combined with a condom, accelerated a water based lubricant's ability to migrate and wet the length of a condom.

1. A composition comprising a powder coated with an aqueous composition comprising a surfactant.

2. The composition of claim 1 wherein said powder is selected from one or more of the group consisting of talc, silica, lycopodium, corn or potato starch, cotton flock, cross-linked nylon or polyurethane, carbonate compounds of carbonates and acetates

3. The composition of claim 1 wherein said surfactant is amphoteric, anionic, cationic, nonionic or a long-chain fatty amine.

4. The composition of claim 3 wherein said aqueous composition comprising a surfactant is a component of a water soluble lubricant.

5. The composition of claim 4 wherein said water soluble lubricant comprises glycerin and a preservative system.

6. The composition of claim 5 wherein said water soluble lubricant is a personal lubricant.

7. The composition of claim 6 wherein said personal lubricant is Astroglide(™).

8. The composition of claim 1 wherein said aqueous composition comprising a surfactant is disposed on a surface of the condom.

9. The composition of claim 8 further comprising an aliquot of water soluble lubricant in contact with a portion of said powder.

10. The composition of claim 9 encased in an airtight container.

11. A condom wherein a surface of said condom is coated with a composition comprising a powder coated with an aqueous composition comprising a surfactant.

12. The condom of claim 11 wherein said powder is selected from one or more of the group consisting of talc, silica, lycopodium, corn or potato starch, cotton flock, cross-linked nylon or polyurethane, carbonate compounds of carbonates and acetates

13. The condom of claim 11 wherein said surfactant is amphoteric, anionic, cationic, nonionic or a long-chain fatty amine.

14. The condom of claim 11 wherein said aqueous composition comprising a surfactant is a component of a water soluble lubricant.

15. The condom of claim 11 wherein said water soluble lubricant comprises glycerin and a preservative system.

16. The condom of claim 11 wherein said water soluble lubricant is a personal lubricant.

17. The condom of claim 11 wherein said personal lubricant is Astroglide(™).

18. The condom of claim 11 further comprising an aliquot of water soluble lubricant in contact with a portion of said powder.

19. The condom of claim 18 encased in an airtight container.

20. A condom comprising a surface which bears a coat of a composition which comprises a powder coated with an aqueous composition comprising a surfactant, and an aliquot of a water soluble lubricant in contact with said powder, said condom encased in an air tight container.

21. A method of making a powder composition, said method comprising the step of mixing a powder with an aqueous composition of surfactant.

22. A method of preparing a condom, said method comprising the step of coating at least one surface of said condom with a composition which comprises a powder coated with an aqueous composition comprising a surfactant, thereby forming a coated surface.

23. The method of claim 22 further comprising the step of depositing an aliquot of a water soluble lubricant on said coated surface.

24. The method of claim 23 further comprising the step of encasing said condom in an airtight container.

25. A method of manufacturing a lubricated condom, said method comprising the steps of:
(a) fabricating a condom;
(b) coating a surface of said condom with a composition which comprises a powder coated with an aqueous composition comprising a surfactant, thereby forming a coated surface;
(c) depositing an aliquot of a water soluble lubricant on said coated surface.

26. The of claim 25 further comprising the step of encasing said condom after step (c) in an airtight container.
